(19) United States
(12) Patent Application Publication
(10) Pub. No.: US 2009/0035781 A1
(43) Pub. Date: Feb. 5, 2009

(54) METHODS AND COMPOSITIONS FOR IDENTIFYING A SUBJECT WITH AN INCREASED RISK OF GRAM NEGATIVE BACTERIAL INFECTION

(75) Inventor: Jason W. Chien, Seattle, WA (US)

Correspondence Address: MYERS BIGEL SIBLEY & SAJOVEC, PO BOX 37428, RALEIGH, NC, 27627, US

(73) Assignee: Fred Hutchinson Cancer Research Center

(21) Appl. No.: 12/183,480

(22) Filed: Jul. 31, 2008

Related U.S. Application Data
(60) Provisional application No. 60/962,665, filed on Jul. 31, 2007.

(51) Int. Cl. C12Q 1/68

(52) U.S. Cl. 435/6

(57) ABSTRACT

The present invention provides method of identifying a subject as having an increased risk of developing a Gram negative bacterial infection and/or as having an increased risk of mortality, comprising genotyping the subject for the presence of particular alleles of the lipopolysaccharide binding protein gene, wherein the presence of said allele(s) identifies the subject as having an increased risk of developing a Gram negative bacterial infection and/or of having an increased risk of mortality.

### STATEMENT OF PRIORITY

This application claims the benefit, under 35 U.S.C. § 119(e), of U.S. Provisional Application No. 60/962,665, filed Jul. 31, 2007, the entire contents of which are incorporated by reference herein.

### GOVERNMENT SUPPORT

Aspects of the present invention were made with the support of federal grant numbers K23HL69860, AI33484, CA15704, CA18029 and HL87690 from the National Institutes of Health. The United States Government has certain rights to this invention.

### FIELD OF THE INVENTION

The present invention provides methods and compositions directed to identification of genetic markers associated with increased risk of Gram negative bacterial infection and/or mortality in a subject, particularly a high risk subject, which can be, e.g., a transplant recipient.

### BACKGROUND OF THE INVENTION

The lethal effects of Gram-negative (GN) bacteria are attributable to lipopolysaccharide (LPS), a highly conserved glycolipid component of the cell wall of all GN bacteria^{1,2}. One of the key components of the innate immune response to LPS is lipopolysaccharide binding protein (LBP), a secretory class 1 acute-phase protein synthesized by hepatocytes. LBP is involved in LPS recognition and signaling³. Circulating LBP can have both pro- and anti-inflammatory effects on the host response to LPS. At low to normal concentrations, LBP catalyzes the transfer of disaggregated LPS to the binding site of membrane-bound and soluble forms of CD14, facilitating signaling via TLR4⁴⁻⁶ and binds directly to GN bacteria, resulting in enhanced phagocytosis and clearance from blood⁷. At high concentrations, LBP can inhibit LPS-induced host cell activation, reduce LPS binding to monocytes, and attenuate the release of proinflammatory cytokines such as TNF-α^{8,9}.

LBP's concentration-dependent immunologic function appears to rely on precise genetic regulation of gene transcription. Thus, genetic variation in the elements controlling LBP production may affect an individual's immune response to LPS and GN bacteria.

The present invention overcomes previous shortcomings in the art by employing a two-stage genetic association study, resulting in the identification of genetic markers in the LBP gene that are associated with an increased risk of Gram negative bacterial infection and/or an increased risk of mortality, particularly in high risk subjects, such as immunocompromised subjects and transplant recipients.

### SUMMARY OF THE INVENTION

In one aspect, the present invention provides a method of identifying a subject as having an increased risk of developing a Gram negative bacterial infection, comprising genotyping the subject for the presence of a C allele of the single nucleotide polymorphism rs2232582 (SNP 6878) of the lipopolysaccharide binding protein gene, wherein the presence of said C allele identifies the subject as having an increased risk of developing a Gram negative bacterial infection.

In another aspect, this invention provides a method of identifying a subject as having an increased risk of developing a Gram negative bacterial infection, comprising genotyping the subject for the presence of a C allele of the single nucleotide polymorphism rs2232571 (SNP 1683) of the lipopolysaccharide binding protein gene, wherein the presence of said C allele identifies the subject as having an increased risk of developing a Gram negative bacterial infection.

In a further aspect of this invention, a method is provided of identifying a subject as having an increased risk of developing a Gram negative bacterial infection, wherein the subject is a high risk subject (e.g., an immunocompromised subject), comprising genotyping the subject for the presence of a C allele of the single nucleotide polymorphism rs2232582 (SNP 6878) of the lipopolysaccharide binding protein gene, wherein the presence of said C allele identifies the subject as having an increased risk of developing a Gram negative bacterial infection.

Also provided herein is a method of identifying a subject as having an increased risk of developing a Gram negative bacterial infection, wherein the subject is a high risk subject (e.g., an immunocompromised subject), comprising genotyping the subject for the presence of a C allele of the single nucleotide polymorphism rs2232571 (SNP 1683) of the lipopolysaccharide binding protein gene, wherein the presence of said C allele identifies the subject as having an increased risk of developing a Gram negative bacterial infection.

An additional aspect of the present invention is a method of identifying a subject as having an increased risk of mortality, comprising genotyping the subject for the presence of a C allele of the single nucleotide polymorphism rs2232582 (SNP 6878) of the lipopolysaccharide binding protein gene, wherein the presence of said C allele identifies the subject as having an increased risk of mortality.

An additional aspect of the present invention is a method of identifying a subject as having an increased risk of mortality, wherein the subject is a high risk subject (e.g., an immunocompromised subject), comprising genotyping the subject for the presence of a C allele of the single nucleotide polymorphism rs2232582 (SNP 6878) of the lipopolysaccharide binding protein gene, wherein the presence of said C allele identifies the subject as having an increased risk of mortality.

Further provided herein is a method of identifying a subject as having an increased risk of mortality, comprising genotyping the subject for the presence of a C allele of the single nucleotide polymorphism rs2232571 (SNP 1683) of the lipopolysaccharide binding protein gene, wherein the presence of said C allele identifies the subject as having an increased risk of mortality.

Further provided herein is a method of identifying a subject as having an increased risk of mortality, wherein the subject is a high risk subject (e.g., an immunocompromised subject), comprising genotyping the subject for the presence of a C allele of the single nucleotide polymorphism rs2232571 (SNP 1683) of the lipopolysaccharide binding protein gene, wherein the presence of said C allele identifies the subject as having an increased risk of mortality.

In additional embodiments, the present invention provides a method of identifying a subject as having an increased risk of developing a Gram negative bacterial infection, comprising genotyping the subject for the presence of an allele of a single nucleotide polymorphism of the lipopolysaccharide binding protein gene of the subject, wherein the allele is selected from the group consisting of: a) a C allele of the single nucleotide polymorphism rs2232571 (SNP 1683); b) a C allele of the single nucleotide polymorphism rs2232582 (SNP 6878); c) a C allele of the single nucleotide polymorphism rs2232575 (SNP 2111); d) a G allele of the single nucleotide polymorphism rs2232578 (SNP 2314); e) an A allele of the single nucleotide polymorphism rs6025049 (SNP 4507); f) a G allele of the single nucleotide polymorphism rs5741813 (SNP 6624); g) a T allele of the single nucleotide polymorphism rs5741814 (SNP 6662); h) a G allele of the single nucleotide polymorphism rs2232581 (SNP 6746); i) a C allele of the single nucleotide polymorphism rs5741815 (SNP 7127); j) a G allele of the single nucleotide polymorphism rs2232590 (SNP 11283); and h) any combination thereof, wherein the presence of said allele or combination of alleles identifies the subject as having an increased risk of developing a Gram negative bacterial infection.

Additionally provided is a method of identifying a subject as having an increased risk of developing a Gram negative bacterial infection, wherein the subject is a high risk subject (e.g., an immunocompromised subject), comprising genotyping the subject for the presence of an allele of a single nucleotide polymorphism of the lipopolysaccharide binding protein gene of the subject, wherein the allele is selected from the group consisting of: a) a C allele of the single nucleotide polymorphism rs2232571; b) a C allele of the single nucleotide polymorphism rs2232582; c) a C allele of the single nucleotide polymorphism rs2232575; d) a G allele of the single nucleotide polymorphism rs2232578; e) an A allele of the single nucleotide polymorphism rs6025049; f) a G allele of the single nucleotide polymorphism rs5741813; g) a T allele of the single nucleotide polymorphism rs5741814; h) a G allele of the single nucleotide polymorphism rs2232581; i) a C allele of the single nucleotide polymorphism rs5741815; j) a G allele of the single nucleotide polymorphism rs2232590; and h) any combination thereof, wherein the presence of said allele or combination of alleles identifies the subject as having an increased risk of developing a Gram negative bacterial infection.

Also provided herein is a method of identifying a subject as having an increased risk of mortality, comprising genotyping the subject for the presence of an allele of a single nucleotide polymorphism of the lipopolysaccharide binding protein gene of the subject, wherein the allele is selected from the group consisting of: a) a C allele of the single nucleotide polymorphism rs2232571; b) a C allele of the single nucleotide polymorphism rs2232582; c) a C allele of the single nucleotide polymorphism rs2232575; d) a G allele of the single nucleotide polymorphism rs2232578; e) an A allele of the single nucleotide polymorphism rs6025049; f) a G allele of the single nucleotide polymorphism rs5741813; g) a T allele of the single nucleotide polymorphism rs5741814; h) a G allele of the single nucleotide polymorphism rs2232581; i) a C allele of the single nucleotide polymorphism rs5741815; j) a G allele of the single nucleotide polymorphism rs2232590; and h) any combination thereof, wherein the presence of said allele or combination of alleles identifies the subject as having an increased risk of mortality.

Also provided herein is a method of identifying a subject as having an increased risk of mortality, wherein the subject is a high risk subject (e.g., an immunocompromised subject), comprising genotyping the subject for the presence of an allele of a single nucleotide polymorphism of the lipopolysaccharide binding protein gene of the subject, wherein the allele is selected from the group consisting of: a) a C allele of the single nucleotide polymorphism rs2232571; b) a C allele of the single nucleotide polymorphism rs2232582; c) a C allele of the single nucleotide polymorphism rs2232575; d) a G allele of the single nucleotide polymorphism rs2232578; e) an A allele of the single nucleotide polymorphism rs6025049; f) a G allele of the single nucleotide polymorphism rs5741813; g) a T allele of the single nucleotide polymorphism rs5741814; h) a G allele of the single nucleotide polymorphism rs2232581; i) a C allele of the single nucleotide polymorphism rs5741815; j) a G allele of the single nucleotide polymorphism rs2232590; and h) any combination thereof, wherein the presence of said allele or combination of alleles identifies the subject as having an increased risk of mortality.

In further aspects, the present invention provides a method of screening for increased risk of a Gram negative bacterial infection or increased mortality (e.g., in a high risk subject), wherein the presence of an allele in the lipopolysaccharide binding protein gene of the subject selected from the group consisting of: a) a C allele of the single nucleotide polymorphism rs2232571; b) a C allele of the single nucleotide polymorphism rs2232582; c) a C allele of the single nucleotide polymorphism rs2232575; d) a G allele of the single nucleotide polymorphism rs2232578; e) an A allele of the single nucleotide polymorphism rs6025049; f) a G allele of the single nucleotide polymorphism rs5741813; g) a T allele of the single nucleotide polymorphism rs5741814; h) a G allele of the single nucleotide polymorphism rs2232581; i) a C allele of the single nucleotide polymorphism rs5741815; j) a G allele of the single nucleotide polymorphism rs2232590; and h) any combination thereof, indicates said subject is at increased risk of a Gram negative bacterial infection or increased mortality, comprising detecting the presence or absence of said allele(s) in a biological sample (e.g., a sample containing nucleic acid) of said subject.

In additional aspects, the present invention provides the use of a means of detecting an allele of a lipopolysaccharide binding protein, wherein said allele is selected from the group consisting of: a) a C allele of the single nucleotide polymorphism rs2232571; b) a C allele of the single nucleotide polymorphism rs2232582; c) a C allele of the single nucleotide polymorphism rs2232575; d) a G allele of the single nucleotide polymorphism rs2232578; e) an A allele of the single nucleotide polymorphism rs6025049; f) a G allele of the single nucleotide polymorphism rs5741813; g) a T allele of the single nucleotide polymorphism rs5741814; h) a G allele of the single nucleotide polymorphism rs2232581; i) a C allele of the single nucleotide polymorphism rs5741815; j) a G allele of the single nucleotide polymorphism rs2232590; and h) any combination thereof, in a biological sample of a subject (e.g., a high risk subject), in determining if said subject is at increased risk of a Gram negative bacterial infection or mortality.

### BRIEF DESCRIPTION OF THE DRAWINGS

FIG. 1. Pairwise analysis of linkage disequilibrium, based upon r², among LBP SNPs. These figures are based upon LBP sequence data provided by the Innate Immunity Program for Genomic Application, which sequenced the entire LBP gene and flanking regions in 23 CEPH European Americans from the Coriell Cell Repository

FIGS. 2A-C. Relationship between SNP 1683, circulating LBP levels, and mortality. FIG. 2A: Heterozygous and homozygous recessive patients had higher median circulating LBP levels measured prior to transplant (boxplot; p=0.004). Box indicates 25^{th} percentile and whiskers, 75^{th} percentile. FIGS. 2B-C: Kaplan-Meier survival curve stratified by whether Gram-negative bacteremia developed (Yes versus No). As more patients with the SNP 1683 C allele (dashed line) died, this proportion was higher among patients who developed Gram-negative bacteremia.

### DETAILED DESCRIPTION OF THE INVENTION

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof.

The present invention is based on the unexpected discovery of the association between certain genetic markers in the LBP gene and Gram negative bacterial infection and/or increased mortality in a subject, which in some embodiments can be a high risk subject such as a subject who is immunocompromised, including a subject who is a transplant recipient.

Thus, in one embodiment, the present invention provides a method of identifying a subject as having an increased risk of developing a Gram negative bacterial infection, comprising genotyping the subject for the presence of a C allele of the single nucleotide polymorphism rs2232582 and/or the presence of a C allele of the single nucleotide polymorphism rs223571 of the lipopolysaccharide binding protein gene, wherein the presence of said C allele(s) identifies the subject as having an increased risk of developing a Gram negative bacterial infection.

In further embodiments, the present invention provides a method of identifying a subject as having an increased risk of developing a Gram negative bacterial infection, wherein the subject is a high risk subject, such as an immunocompromised subject, comprising genotyping the subject for the presence of a C allele of the single nucleotide polymorphism rs2232582 and/or the presence of a C allele of the single nucleotide polymorphism rs223571 of the lipopolysaccharide binding protein gene, wherein the presence of said C allele(s) identifies the subject as having an increased risk of developing a Gram negative bacterial infection.

In additional embodiments, the present invention provides a method of identifying a subject as having an increased risk of mortality, comprising genotyping the subject for the presence of a C allele of the single nucleotide polymorphism rs2232582 and/or the C allele of the single nucleotide polymorphism rs223571 of the lipopolysaccharide binding protein gene, wherein the presence of said C allele(s) identifies the subject as having an increased risk of mortality. In certain embodiments of this method, the subject can be a high risk subject.

In other embodiments of this invention, provided herein is a method of identifying a subject, which in some embodiments can be a high risk subject, such as an immunocompromised subject, as having an increased risk of developing a Gram negative bacterial infection, comprising genotyping the subject for the presence of an allele of a single nucleotide polymorphism of the lipopolysaccharide binding protein gene of the subject, wherein the allele is selected from the group consisting of: a) a C allele of the single nucleotide polymorphism rs2232571; b) a C allele of the single nucleotide polymorphism rs2232582; c) a C allele of the single nucleotide polymorphism rs2232575; d) a G allele of the single nucleotide polymorphism rs2232578; e) an A allele of the single nucleotide polymorphism rs6025049; f) a G allele of the single nucleotide polymorphism rs5741813; g) a T allele of the single nucleotide polymorphism rs5741814; h) a G allele of the single nucleotide polymorphism rs2232581; i) a C allele of the single nucleotide polymorphism rs5741815; j) a G allele of the single nucleotide polymorphism rs2232590; and h) any combination thereof, wherein the presence of said allele or combination of alleles identifies the subject as having an increased risk of developing a Gram negative bacterial infection.

Yet further embodiments include a method of identifying a subject as having an increased risk of mortality, which can be a high risk subject, such as an immunocompromised subject, comprising genotyping the subject for the presence of an allele of a single nucleotide polymorphism of the lipopolysaccharide binding protein gene of the subject, wherein the allele is selected from the group consisting of: a) a C allele of the single nucleotide polymorphism rs2232571; b) a C allele of the single nucleotide polymorphism rs2232582; c) a C allele of the single nucleotide polymorphism rs2232575; d) a G allele of the single nucleotide polymorphism rs2232578; e) an A allele of the single nucleotide polymorphism rs6025049; f) a G allele of the single nucleotide polymorphism rs5741813; g) a T allele of the single nucleotide polymorphism rs5741814; h) a G allele of the single nucleotide polymorphism rs2232581; i) a C allele of the single nucleotide polymorphism rs5741815; j) a G allele of the single nucleotide polymorphism rs2232590; and h) any combination thereof, wherein the presence of said allele or combination of alleles identifies the subject as having an increased risk of mortality.

An LBP allele of this invention is a C allele of the single nucleotide polymorphism rs2232571 (SNP 1683); a C allele of the single nucleotide polymorphism rs2232582 (SNP 6878); a C allele of the single nucleotide polymorphism rs2232575 (SNP 2111); a G allele of the single nucleotide polymorphism rs2232578 (SNP 2314); an A allele of the single nucleotide polymorphism rs6025049 (SNP 4507); a G allele of the single nucleotide polymorphism rs5741813 (SNP 6624); a T allele of the single nucleotide polymorphism rs5741814 (SNP 6662); a G allele of the single nucleotide polymorphism rs2232581 (SNP 6746); a C allele of the single nucleotide polymorphism rs5741815 (SNP 7127); a G allele of the single nucleotide polymorphism rs2232590 (SNP 11283); and any combination thereof.

The present invention further provides a method of identifying a subject at increased risk of developing a Gram negative bacterial infection and/or increased risk of mortality, comprising genotyping the subject for the presence of a G allele of the single nucleotide polymorphism rs2232596 (SNP 17002), wherein the presence of said allele identifies the subject as having an increased risk of developing a Gram negative bacterial infection and/or having an increased risk of mortality. The subject of this method can be a high risk subject. Furthermore, this method employing SNP17002 can be combined with methods employing SNPs of BIN B1 in any combination to identify subjects of this invention as having an increased risk of developing a Gram negative bacterial infection and/or having an increased risk of mortality

A subject of this invention can have one copy of an LBP allele of this invention and be heterozygous for the particular allele or the subject can have two copies of an LBP allele of this invention and be homozygous for the particular allele. A subject of this invention can be heterozygous for the alleles of this invention and/or homozygous for the alleles of this invention in any combination.

A subject of this invention can be any subject susceptible to Gram negative bacterial infection. Thus, the subject can be any animal that is susceptible to Gram negative bacterial infection and in particular embodiments, the subject can be a human. The subject can also be of any race or ethnic origin, including whites, Caucasians, blacks, African Americans, Hispanics and Asians.

The Gram negative bacterial infection of this invention can be caused by any Gram negative bacterium now known, or later identified, to cause infection in animals, including but not limited to, Acinetobacter species, Aeromonas species, Agrobacterium tumefaciens, Alcaligenes species, Bacteroides species, Burkholderia species, Citrobacter species, Enterobacter species, Escherichia coli, Klebsiella species, Leptotrichia species, Morganella species, Moraxella species, Neisseria species, Pantoea species, Proteus species, Pseudomonas species, Ralstonia species, Serratia species, Stenotrophomonas species and any combination thereof.

A subject of this invention can be a “high risk” subject, which means the subject is immunodeficient or immunocompromised and/or undergoing, contemplating, anticipating, expected to have and/or at risk of having, a stressful event and/or the subject is more vulnerable than average (e.g., more vulnerable than a subject who is not immunodeficient or immunocompromised or not undergoing, contemplating, anticipating, expected to have and/or at risk of having, a stressful event a stressful event as described herein) to a Gram negative bacterial infection and/or mortality due to the subject's immune status and/or one or more stressful events as described herein.

The terms “immunodeficient” and “immunocompromised” as used herein are intended to have their art-recognized meaning as describing a subject whose immune system is impaired or weakened and/or functioning abnormally as compared with a healthy or normal subject. An immunodeficiency or immunocompromised state in a subject can be due to a variety of causes that are well known in the art, including but not limited to, genetic disorders of the immune system, diseases, disorders and/or infections that affect the immune system (e.g., human immunodeficiency virus infection and other viral, parasitic and bacterial infections), autoimmune disorders, drug-induced and/or radiation-induced immunosuppression for transplantation and/or to treat various diseases and disorders, steroid use, chemotherapy, radiation therapy and any other therapies that deplete immune cells, splenectomy, cystic fibrosis, sepsis, cancer, kidney failure, alcoholism, cirrhosis, diabetes, pregnancy, old age, infancy, hypothermia, severe emotional trauma or stress, malnutrition, etc., as would be known in the art.

Nonlimiting examples of a stressful event that would identify a subject as “high risk” include placement in a hospital or other medical facility, an elective surgery, a non-elective surgery, an elective invasive procedure, a non-elective invasive procedure, trauma or injury to the subject (e.g., automobile accident, burn, cold exposure, heat exposure and/or other accidental trauma or injury), emotional and/or psychological stress and/or trauma and/or any disease condition or pathological state that can increase the likelihood of development of a Gram negative bacterial infection in the subject and/or increase the likelihood of mortality, as would be well known to one of skill in the art.

Thus, for example, in some embodiments of the invention, the subject can be a perioperative patient, a postoperative patient, a preoperative patient, a periprocedural patient, a postprocedural patient, a preprocedural patient, an intensive care unit patient, a post-intensive care unit patient, a trauma patient, an acutely ill patient, a chronically ill patient, a mentally ill patient, a patient with a psychological and/or emotional disorder and any combination of the above.

Further, a subject of this invention can be a subject who is about to undergo a surgery and/or invasive procedure, a subject who is preparing to undergo a surgery and/or invasive procedure and/or a subject who is about to undergo and/or is preparing to undergo a medical treatment that can increase the likelihood of the development of a Gram negative bacterial infection in the subject and/or increase the likelihood of mortality in the subject. In some embodiments, the subject of this invention can be a subject who has undergone a surgery and/or invasive procedure and/or a subject who has undergone a medical treatment that can increase the likelihood of development-of a Gram negative bacterial infection in the subject and/or increase the likelihood of mortality in the subject. In addition, the subject of this invention can be a subject who is about to receive and/or who has received medical treatment that does result and/or could result in placement of the subject in an intensive care unit.

As used herein, the terms “perioperative” and “periprocedural” mean the period of time extending from when the subject goes into a hospital, clinic, doctor's office or other facility for surgery, for a procedure and/or for other medical treatment until the time the subject returns home. Accordingly, “preoperative” and “preprocedural” mean the period of time before the subject goes into a hospital, clinic, doctor's office or other facility for surgery, for a procedure and/or for other medical treatment and “postoperative” and “postprocedure” mean the period of time after the subject returns home following the surgery, procedure and/or other medical treatment.

Furthermore, as used herein, “an intensive care unit patient” is a subject who has been admitted to an intensive care unit of a hospital, clinic or other medical facility for any medical condition that warrants intensive care, as would be known by one of skill in the art. A “post-intensive care unit patient” is a subject who had previously been cared for in an intensive care unit of a hospital, clinic or other medical facility and has been discharged from the intensive care unit.

Also as used herein, the term “invasive procedure” means any technique where entry into a body cavity is required or where the normal function of the body is in some way interrupted. An invasive procedure can also be a medical procedure and/or treatment that invades (enters) the body, usually by cutting or puncturing the skin or by inserting instruments into the body.

Nonlimiting examples of an invasive procedure of this invention include endoscopy, bronchoscopy, cardiac catheterization, angioplasty, colonoscopy, hemodialysis, blood transfusion, blood donation, plasma donation, leukopheresis and any combination thereof.

In addition, nonlimiting examples of a surgery, operation or surgical procedure of this invention include transplantation of an organ or tissue (e.g., hematopoietic cells, hematopoietic stem cells, kidney, skin graft, bone graft, liver, heart, heart valve, lung, pancreas, islet cells, intestines cornea, hand, foot, etc.), surgery on an organ or tissue (e.g., heart, lung, stomach, kidneys, uterus, ovaries, intestines, colon, brain, prostate, gall bladder, appendix, joint, etc.), removal of organs, bariatric surgery, laparoscopic surgery, hernia surgery, hemorrhoid surgery, plastic surgery, exploratory surgery, varicose vein surgery, minimally invasive surgery, etc.

It is further contemplated that the methods of this invention can be carried out at any time relative to the event that increases the likelihood of development of a Gram negative bacterial infection and/or increases the likelihood of mortality in the subject. Thus, the methods of this invention can be carried out prior to, during and/or after surgery, an invasive procedure, a trauma or injury and/or a treatment that increases the likelihood of Gram negative bacterial infection and/or mortality. For example, in some embodiments, the methods of this invention can also be carried out prior to, during and/or after a subject is a patient in an intensive care unit.

In further embodiments, the methods of this invention can be carried out on a subject who has developed a Gram negative bacterial infection, including a current infection, as well as a past incident of infection from which the subject has recovered. In additional embodiments, the subject can have a relative (e.g., parent, sibling, child, aunt, uncle, grandparent, niece, nephew, etc.) who has developed a Gram negative bacterial infection, which can be a current infection and/or a past incident of infection.

An allele of the LBP gene is correlated with an increased risk of developing a Gram negative infection and/or with an increased risk of mortality by identifying or detecting the presence of a particular LBP allele in the nucleic acid of subjects also identified as having Gram negative bacterial infection and/or who have died and performing a statistical analysis of the association of the particular LBP allele with the presence of Gram negative bacterial infection and/or mortality in the subject, according to well known methods of statistical analysis. An analysis that identifies a statistical association (e.g., a significant association) between the particular LBP allele and the presence of Gram negative bacterial infection and/or death establishes a correlation between the presence of the LBP allele in the subject and an increased risk of developing a Gram negative bacterial infection and/or increased risk of mortality.

For example, the identification and/or detection of an LBP allele of this invention in a sample (e.g., a biological sample such as blood, cells, tissue, fluid or other sample containing nucleic acid) can be determined using any of a variety of genotyping techniques known in the art, as described below. As used herein, the terms “genotype” or genotyping” mean to examine a nucleic acid sample of a subject (e.g., “test” the sample) to identify the genetic makeup of the subject, i.e., what specific alleles are present in a nucleic acid sample from a subject and/or to detect specific alleles in nucleic acid of the subject. In particular, a subject of the present invention is genotyped to identify which alleles are present in the LBP gene of the subject in order to determine if the subject is at increased risk of a Gram negative bacterial infection and/or increased risk of mortality due to the presence in the LBP gene of the subject of an allele of this invention that has been identified to be associated with increased risk of Gram negative bacterial infection and/or increased risk of mortality.

Thus, the present invention also provides a method of identifying a human subject having an increased risk of a Gram negative bacterial infection, comprising: a) correlating the presence of an allele of a single nucleotide polymorphism in the LBP gene with the presence of a Gram negative bacterial infection; and b) detecting the allele of the single nucleotide polymorphism of step (a) in the subject, thereby identifying a subject having increased risk of Gram negative bacterial infection.

Also provided herein is a method of identifying a single nucleotide polymorphism in the LBP gene correlated with increased risk of Gram negative infection, comprising: a) identifying a subject having a Gram negative bacterial infection; b) detecting in a population of the subjects of (a) above the presence of an allele of a single nucleotide polymorphism in the LBP gene; and c) correlating the presence of the allele of the single nucleotide polymorphism of step (b) with the Gram negative bacterial infection in population of subjects, thereby identifying an allele in the single nucleotide polymorphism in the LBP gene correlated with increased risk of Gram negative bacterial infection.

In additional embodiments, the present invention provides a method of correlating an allele of a single nucleotide polymorphism in the LBP gene of a subject with increased risk of Gram negative bacterial infection, comprising: a) identifying a subject having a Gram negative bacterial infection; b) determining the nucleotide sequence of the LBP gene in a population of the subjects of (a); c) comparing the nucleotide sequence of step (b) with the nucleotide sequence of the LBP gene of a population of subjects without a Gram negative bacterial infection; d) identifying an allele of a single nucleotide polymorphism in the nucleotide sequence of (b) that occurs more frequently than in the nucleotide sequence of the population of subjects without a Gram negative bacterial infection; and e) correlating the allele of the single nucleotide polymorphism of (d) with the presence of Gram negative bacterial infection in the population of subjects of (a), thereby correlating an allele of a single nucleotide polymorphism in the LBP gene of the subject with increased risk of Gram negative bacterial infection.

The present invention also provides a method of screening for an allele in a single nucleotide polymorphism in the LBP gene of a human subject that is associated with increased risk of Gram negative bacterial infection, comprising: a) detecting the alleles of single nucleotide polymorphisms in the LPB gene of a human subject; b) performing a population based study to detect the alleles of (a) in a group of human subjects with Gram negative bacterial infection and ethnically matched controls; and c) identifying an allele of a single nucleotide polymorphism in the LBP gene that is associated with increased risk of Gram negative bacterial infection.

For the methods of this invention, the genotyping of nucleic acid, as well as the detection of an allele in the LBP gene of this invention (GenBank® Accession Nos. NC_{—}000020; NM_{—}004139) can be carried out according to various protocols standard in the art for identifying specific nucleotides in a nucleotide sequence, and as described in the Examples section provided herein.

For example, nucleic acid can be obtained from any suitable sample from the subject that will contain nucleic acid and the nucleic acid can then be prepared and analyzed according to well-established protocols for the presence of genetic markers according to the methods of this invention. In some embodiments, analysis of the nucleic acid can be carried by amplification of the region of interest according to amplification protocols well known in the art (e.g., polymerase chain reaction, ligase chain reaction, strand displacement amplification, transcription-based amplification, self-sustained sequence replication (3SR), Qβ replicase protocols, nucleic acid sequence-based amplification (NASBA), repair chain reaction (RCR) and boomerang DNA amplification (BDA), etc.). The amplification product can then be visualized directly in a gel by staining or the product can be detected by hybridization with a detectable probe. When amplification conditions allow for amplification of all allelic types of a genetic marker, the types can be distinguished by a variety of well-known methods, such as hybridization with an allele-specific probe, secondary amplification with allele-specific primers, by restriction endonuclease digestion, and/or by electrophoresis. Thus, the present invention further provides oligonucleotides (e.g., that are complementary to the nucleotide sequence of the LBP gene and/or coding sequence) for use as primers and/or probes for detecting and/or identifying genetic markers according to the methods of this invention.

The genetic markers of this invention are correlated with Gram negative bacterial infection and/or mortality as described herein according to methods well known in the art and as disclosed in the Examples provided herein for correlating genetic markers with various phenotypic traits, including disease states and pathological conditions and levels of risk associated with developing a disease or pathological condition. In general, identifying such correlation involves conducting analyses that establish a statistically significant association and/or a statistically significant correlation between the presence of a genetic marker or a combination of markers and the phenotypic trait in the subject. An analysis that identifies a statistical association (e.g., a significant association) between the marker or combination of markers and the phenotype establishes a correlation between the presence of the marker or combination of markers in a subject and the particular phenotype being analyzed. Such a statistically significant association can then be used to identify subjects at increased or decreased risk of developing a disease or pathological condition by genotyping nucleic acid of the subject to detect the presence of the associated marker or combination of markers.

The present invention further provides kits suitable for use in identifying an LBP allele of this invention in a nucleic acid sample. Such kits can include, for example, reagents (e.g., probes or primers) necessary to carry out genotyping, as are well known in the art.

In carrying out the methods of this invention, detection reagents can be developed and used to identify any allele of the present invention individually or in combination with the identification of other alleles, and such detection reagents can be readily incorporated into one of the established kit or system formats that are well known in the art. The terms “kits” and “systems,” as used herein refer, e.g., to reagents for detection of a single or multiple alleles, or reagents for detection of one or more alleles in combination with one or more other types of kit or system elements or components (e.g., other types of biochemical reagents, containers, packages such as packaging intended for commercial sale, substrates to which allele detection reagents are attached, electronic hardware components, etc.) Accordingly, the present invention further provides allele detection/identification kits and systems, including but not limited to, packaged probe and primer sets (e.g., TAQMAN® probe/primer sets), arrays/microarrays of nucleic acid molecules, and/or beads that contain one or more probes, primers, and/or other detection reagents for detecting/identifying one or more alleles of the present invention. The kits/systems can optionally include various electronic hardware components; for example, arrays (“DNA chips”) and microfluidic systems (“lab-on-a-chip” systems) provided by various manufacturers. Other kits/systems (e.g., probe/primer sets) may not include electronic hardware components, but can comprise, for example, one or more detection reagents (along with, optionally, other biochemical reagents) packaged in one or more containers.

In some embodiments, a kit of this invention typically contains one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, etc.) necessary to carry out an assay or reaction, such as amplification and/or detection of an allele-containing nucleic acid molecule. In some embodiments of the present invention, kits are provided that contain the necessary reagents to carry out one or more assays to detect one or more alleles disclosed herein. In some embodiments of the present invention, allele detection kits/systems are in the form of nucleic acid arrays, or compartmentalized kits, including microfluidic/lab-on-a-chip systems.

Allele detection kits/systems of this invention can contain, for example, one or more probes, or pairs of probes, that hybridize to a nucleic acid molecule at or near each target allele position. Multiple pairs of allele-specific probes can be included in the kit/system to simultaneously assay large numbers of alleles, at least one of which is an allele of the present invention. In some kits/systems, the allele-specific probes can be immobilized to a substrate such as an array or bead. The terms “arrays,” “microarrays,” and “DNA chips” are used herein interchangeably to refer to an array of distinct polynucleotides affixed to a substrate, such as glass, plastic, paper, nylon and/or other type of membrane, filter, chip, and/or any other suitable solid support. The polynucleotides can be synthesized directly on the substrate, or synthesized separate from the substrate and then affixed to the substrate. In one embodiment, the microarray can be prepared and used according to the methods described, e.g., in U.S. Pat. No. 5,837,832, U.S. Pat. No. 5,807,522, PCT Publication No. WO 95/11995, Lockhart et al. (1996) Nat. Biotech. 14:1675-1680; and Schena et al. (1996) Proc. Nati. Acad. Sci. 93:10614-10619, all of which are incorporated herein in their entireties by reference.

Any number of probes, such as allele-specific probes, can be implemented in an array, and each probe or pair of probes can hybridize to a different allele position. In some embodiments, polynucleotide probes can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized (e.g., to the size of a dime). Preferably, probes are attached to a solid support in an ordered, addressable array.

A microarray can be composed of a large number of unique, single-stranded polynucleotides, usually either synthetic antisense polynucleotides or fragments of cDNAs fixed to a solid support. Exemplary polynucleotides can be about 6-100 nucleotides in length in some embodiments, about 15-30 nucleotides in length in other embodiments, and about 18-25 nucleotides in length in yet other embodiments of this invention. For certain types of microarrays or other detection kits/systems, oligonucleotides that are only about 7-20 nucleotides in length can be used. In other types of arrays, such as arrays used in conjunction with chemiluminescence detection technology, probe lengths can be, for example, about 15-80 nucleotides, about 50-70 nucleotides in length, about 55-65 nucleotides in length, and/or about 60 nucleotides in length. The microarray or detection kit can contain polynucleotides that cover the known 5′ or 3′ sequence of a gene/transcript or target allele site, sequential polynucleotides that cover the full-length sequence of a gene/transcript; and/or unique polynucleotides selected from particular areas along the length of a target gene/transcript sequence.

Hybridization assays based on polynucleotide arrays rely on the differences in hybridization stability of the probes to perfectly or nearly perfectly matched and mismatched target sequence variants. For SNP genotyping, stringency conditions used in hybridization assays can be high enough such that nucleic acid molecules that differ from one another at as little as a single SNP position can be differentiated (e.g., typical SNP hybridization assays are designed so that hybridization will occur only if one particular nucleotide is present at a SNP position, but will not occur if an alternative nucleotide is present at that SNP position). Such high stringency conditions can be used, for example, in nucleic acid arrays of allele-specific probes for SNP detection. Such high stringency conditions are well known to those skilled in the art and can be found in, for example, Current Protocols in Molecular Biology, John Wiley & Sons, N.Y. (1989).

In other embodiments, the arrays are used in conjunction with chemiluminescence detection technology, as is known in the art (see, e.g. U.S. Pat. Nos. 6,124,478, 6,107,024, 5,994,073, 5,981,768, 5,871,938, 5,843,681, 5,800,999, and 5,773,628, which describe methods and compositions for performing chemiluminescence detection; and USPTO Publication No. 2002/0110828, which discloses methods and compositions for microarray controls. All of these references are incorporated herein in their entireties by reference.).

A polynucleotide probe can be synthesized on the surface of the substrate by using a chemical coupling procedure and an ink jet application apparatus, as described, for example, in PCT Publication No. WO 95/251116, which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical linking procedures and/or chemical bonding procedures. An array, such as described above, can be produced by hand or by using available devices (slot blot or dot blot apparatus), materials (any suitable solid support), and/or machines (including robotic instruments), and may contain, e.g., 8, 24, 96, 384, 1536, 6144 or more polynucleotides, or any other number which lends itself to the efficient use of commercially available instrumentation.

Using such arrays and/or other kits/systems, the present invention provides methods of identifying and/or detecting the alleles disclosed herein in a biological test sample. Such methods typically involve incubating a sample containing nucleic acid with an array comprising one or more probes corresponding to at least one allele of the present invention, and assaying for binding of a nucleic acid from the test sample with one or more of the probes. Conditions for incubating a detection reagent (or a kit/system that employs one or more such detection reagents) with a test sample vary. Incubation conditions depend on such factors as the format employed in the assay, the detection methods employed, and/or the type and nature of the detection reagents used in the assay. One skilled in the art will recognize that any one of the commonly available hybridization, amplification and array assay formats can readily be adapted to detect the alleles of this invention as disclosed herein.

A detection kit/system of the present invention can include components that are used to prepare nucleic acids from a test sample for the subsequent amplification and/or detection of an allele-containing nucleic acid molecule. Such sample preparation components can be used to produce nucleic acid extracts (including DNA and/or RNA), proteins or membrane extracts from any bodily fluids (such as blood, serum, plasma, urine, saliva, phlegm, gastric juices, semen, tears, sweat, etc.), skin, hair, cells (especially nucleated cells), biopsies, buccal swabs or tissue specimens. The test samples used in the above-described methods will vary based on such factors as the assay format, nature of the detection method, and the specific tissues, cells or extracts used as the test sample to be assayed. Methods of preparing nucleic acids, proteins, and cell extracts are well known in the art and can be readily adapted to obtain a sample that is compatible with the system utilized. Automated sample preparation systems for extracting nucleic acids from a test sample are commercially available (e.g., Qiagen's BIOROBOT 9600® system, Applied Biosystems' PRISM 6700® system, and Roche Molecular Systems COBAS AmpliPrep® system).

Another form of kit included in the present invention is a compartmentalized kit. A compartmentalized kit includes any kit in which reagents are contained in separate containers. Such containers include, for example, small glass containers, plastic containers, strips of plastic, glass and/or paper, and/or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other allele detection reagent for detecting one or more alleles of the present invention, one or more containers which contain wash reagents (such as phosphate buffered saline, Tris-buffers, etc.), and one or more containers which contain the reagents used to reveal the presence of the bound probe or other allele detection reagents. The kit can optionally further comprise compartments and/or reagents for, for example, nucleic acid amplification or other enzymatic reactions such as primer extension reactions, hybridization, ligation, electrophoresis (preferably capillary electrophoresis), mass spectrometry, and/or laser-induced fluorescence detection. The kit can also include instructions for using the kit. Exemplary compartmentalized kits include microfluidic devices known in the art (e.g., Weigl et al. (2003) “Lab-on-a-chip for drug development” Adv Drug Deliv Rev. 55(3):349-77). In such microfluidic devices, the containers may be referred to as, for example, microfluidic “compartments,” “chambers,” or “channels.”

Microfluidic devices, which may also be referred to as “lab-on-a-chip” systems, biomedical micro-electro-mechanical systems (bioMEMs), or multicomponent integrated systems, are exemplary kits/systems of the present invention for analyzing nucleic acid (e.g., detecting specific alleles). Such systems miniaturize and compartmentalize processes such as probe/target hybridization, nucleic acid amplification, and capillary electrophoresis reactions in a single functional device. Such microfluidic devices typically utilize detection reagents in at least one aspect of the system, and such detection reagents may be used to detect one or more alleles of the present invention. One example of a microfluidic system is disclosed in U.S. Pat. No. 5,589,136, which describes the integration of PCR-amplification and capillary electrophoresis in chips and which is incorporated by reference herein in its entirety. Exemplary microfluidic systems comprise a pattern of microchannels designed onto a glass, silicon, quartz, or plastic wafer included on a microchip. The movements of the samples can be controlled by electric, electroosmotic and/or hydrostatic forces applied across different areas of the microchip to create functional microscopic valves and pumps with no moving parts. Varying the voltage can be used as a means to control the liquid flow at intersections between the micro-machined channels and/or to change the liquid flow rate for pumping across different sections of the microchip. See, for example, U.S. Pat. No. 6,153,073 and U.S. Pat. No. 6,156,181.

For genotyping alleles of this invention, an exemplary microfluidic system may integrate, for example, nucleic acid amplification, primer-extension, capillary electrophoresis, and a detection method such as laser induced fluorescence detection. In a first step of such an exemplary system, nucleic acid samples are amplified, preferably by PCR. Then, the amplification products are subjected to automated primer extension reactions using ddNTPs (employing specific fluorescence for each ddNTP) and the appropriate oligonucleotide primers to carry out primer extension reactions that hybridize just upstream of the targeted allele. Once the extension at the 3′ end is completed, the primers are separated from the unincorporated fluorescence ddNTPs by capillary electrophoresis. The separation medium used in capillary electrophoresis can be, for example, polyacrylamide, polyethyleneglycol or dextran. The incorporated ddNTPs in the single nucleotide primer extension products are identified by laser-induced fluorescence detection. Such an exemplary microchip can be used to process, for example, at least 96 to 384 samples, or more, in parallel.

As noted above, any of a variety of suitable techniques can be employed in the methods of this invention for detection of an allele of this invention. Such techniques can include, for example, the use of microsatellite array analysis, restriction fragment length polymorphism (RFLP) analysis, mass spectrometry (Ye et al., Hum. Mutat. 17(4):305 (2001); Chen et al., Genome Res. 10:549 (2000)), nanotechnology protocols for genomic characterization and/or any other protocol or technique now known or later developed for use in identifying genomic characteristics, including any of a variety of single nucleotide polymorphism (SNP) detection techniques now known or later developed.

In particular, for the identification of single-nucleotide polymorphisms (SNPs) in nucleic acid, various methods can be used, including, but not limited to, fluorescence-based sequencing, hybridization high-density variation-detection DNA chips, high performance liquid chromatography, allele-specific oligonucleotide hybridization (ASOH), nick translation PCR, PCR-ELISA ASO I0 typing, dynamic allele-specific hybridization (DASH), allele-specific inverse PCR (ASIP), inverse PCR-RFLP (IP-RFLP), single stranded conformational polymorphism (SSCP) genotyping, bi-directional PCR amplification of specific allele (bi-PASA), high-throughput SNP genotyping, homogeneous allele-specific PCR based SNP genotyping, molecular inversion probe genotyping, amplification refractory mutation system (ARMS), locked nucleic (LN) SNP genotyping, molecular beacon sequence analysis, high performance multiplex SNP analysis, amplified fragment length polymorphism (AFLP), melting curve analysis of SNPs, tetra-primer ARMS-PCR, ligase chain reaction, allele-specific polymerase chain reaction; Tₘ shift genotyping, and/or minisequencing.

“Single Nucleotide Polymorphism” or “SNP” refers to single-base pair variations within the genetic code of the individuals of a population. SNPs, which are defined in relation to a population, are variations in DNA at a single base that are found in at least 1% of the population. The terms “biallelic marker,” “marker,” “polymorphism” and “allele” are also used to denote variations at a single base and are used interchangeably. SNPs and other alleles can be identified de novo through population analysis or can be selected from numerous databases including the National Center for Biotechnology Information (NCBI) SNP database (dbSNP), the SNP Consortium (TSC) database, Human Genome Variation Database (HGVbase), and the ABI database (Applied Biosystems, Foster City, Calif.).

The term “genotype” is used herein to refer to a specific allele or combination of alleles that an individual carries at a given locus. It can also be used to describe a set of alleles for multiple loci.

Also as used herein, a “haplotype” refers to a set of alleles on a single chromatid that are statistically associated. It is thought that these associations, and the identification of a few alleles of a haplotype block, can unambiguously identify most other polymorphic sites in its region. Such information is very valuable for investigating the genetics behind common diseases and is collected by the International HapMap Project. The term “haplotype” is also commonly used to describe the genetic constitution of individuals with respect to one member of a pair of allelic genes; sets of single alleles or closely linked genes that tend to be inherited together.

The term “phenotype” is used herein to mean the form taken by some character (or group of characters) in a specific individual. It can also mean the detectable outward manifestations of a specific genotype.

An “allele” as used herein refers to one of two or more alternative forms of a nucleotide sequence at a given position (locus) on a chromosome. Usually alleles are nucleotide sequences that make up the coding sequence of a gene, but sometimes the term is used to refer to a nucleotide sequence in a non-coding sequence. An individual's genotype for a given gene is the set of alleles it happens to possess.

The term “allele frequency” is used herein to refer to a measure of the commonness of an allele in a population; the proportion of all alleles of that gene or polymorphism in the population that are of this specific type.

The term “Hardy-Weinberg” is used to refer to calculating the Hardy-Weinberg equilibrium for genotypes, wherein the stable frequency distribution of genotypes AA, Aa, and aa, in the proportions p², 2pq and q², respectively (where p and q are the frequencies of the alleles A and a) is determined, which is a consequence of random mating in the absence of mutation, migration, natural selection or random drift.

The term “p-value” is used herein to describe the probability that the results are not significant. For example, a p-value of 0.05 means that there are 5 chances in 100 that the results are not significant.

The term “SEM” is used to mean the standard of the mean.

The term “linkage disequilibrium” is used herein to refer to the relationship that is said to exist between an allele found at a single polymorphic site and alleles found at nearby polymorphisms if the presence of one allele is strongly predictive of the alleles present at the nearby polymorphic sites. Thus, the existence of linkage disequilibrium (LD) enables an allele of one polymorphic marker to be used as a surrogate for a specific allele of another. Furthermore, as used herein, the term “linkage disequilibrium” or “LD” refers to the occurrence in a population of two linked alleles at a frequency higher or lower than expected on the basis of the allele frequencies of the individual genes. Thus, linkage disequilibrium describes a situation where alleles occur together more often than can be accounted for by chance, which indicates that the two alleles are physically close on a DNA strand.

The term “polynucleotide” refers to a chain of nucleotides without regard to length of the chain.

Also as used herein, “linked” describes a region of a chromosome that is shared more frequently in family members or members of a population affected by a particular disease or disorder, than would be expected or observed by chance, thereby indicating that the gene or genes or other identified marker(s) within the linked chromosome region contain or are associated with an allele that is correlated with the presence of a disease or disorder, or with an increased or decreased risk of the disease or disorder. Once linkage is established, association studies (linkage disequilibrium) can be used to narrow the region of interest or to identify the marker (e.g., allele or haplotype) correlated with the disease or disorder.

The term “genetic marker” as used herein refers to a region of a nucleotide sequence (e.g., in a chromosome) that is subject to variability (i.e., the region can be polymorphic for a variety of alleles). For example, a single nucleotide polymorphism (SNP) in a nucleotide sequence is a genetic marker that is polymorphic for two (or in some cases, three or four) alleles. SNPs can be present within a coding sequence of a gene, within noncoding regions of a gene and/or in an intergenic (e.g., intron) region of a gene. A SNP in a coding region in which both allelic forms lead to the same polypeptide sequence is termed synonymous (i.e., a silent mutation) and if a different polypeptide sequence is produced, the alleles of that SNP are non-synonymous. SNPs that are not in protein coding regions can still have effects on gene splicing, transcription factor binding and/or the sequence of the non-coding RNA.

Other examples of genetic markers of this invention can include but are not limited to haplotypes (i.e., combinations of alleles), microsatellites, restriction fragment length polymorphisms (RFLPs), repeats (i.e., duplications), insertions, deletions, etc., as are well known in the art.

As used herein, “nucleic acids” encompass both RNA and DNA, including cDNA, genomic DNA, mRNA, synthetic (e.g., chemically synthesized) DNA and chimeras of RNA and DNA. The nucleic acid can be double-stranded (i.e., the sequence and its complementary sequence) or single-stranded. Where single-stranded, the nucleic acid can be a sense strand or an antisense strand. The nucleic acid can be synthesized using nucleotide analogs or derivatives (e.g., inosine or phosphorothioate nucleotides). Such nucleotides can be used, for example, to prepare nucleic acids that have altered base-pairing abilities or increased resistance to nucleases.

An “isolated nucleic acid” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence.

The term “isolated” can refer to a nucleic acid or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state.

The term “oligonucleotide” refers to a nucleic acid sequence of at least about six nucleotides to about 100 nucleotides, for example, about 15 to 30 nucleotides, or about 20 to 25 nucleotides, which can be used, for example, as a primer in a PCR amplification or as a probe in a hybridization assay or in a microarray. Oligonucleotides can be natural or synthetic, e.g., DNA, RNA, modified backbones, etc. Peptide nucleic acids (PNAs) can also be used as probes in the methods of this invention.

The present invention further provides a method of identifying an effective treatment regimen for a subject with a Gram negative bacterial infection, comprising correlating the presence of one or more alleles of the LBP gene of this invention with an effective treatment regimen for a Gram negative bacterial infection.

Thus, the methods of this invention can be used to identify subjects most suited to therapy with particular pharmaceutical agents, e.g., to prophylactically treat a subject at increased risk of developing a Gram negative bacterial infection and/or at increased risk of mortality. Thus, the present invention further provides a method of identifying a patient in need of such prophylactic treatment, comprising detecting an LBP allele of this invention in the subject. Similarly, the identification of an LBP allele of this invention in a subject can be used to exclude patients from certain surgeries, procedures and/or treatments due to the patient's increased likelihood of developing a Gram negative bacterial infection and/or increased likelihood of mortality.

Thus, in further embodiments, the present invention provides a method of identifying a subject who is not suitable for surgery, an invasive procedure, a transplant and/or a treatment that increases the likelihood of the development of Gram negative bacterial infection and/or mortality in the subject, comprising detecting an LBP allele of this invention in the subject. The methods of this invention can also be employed in other pharmacogenomics analyses to assist the drug development and selection process. (Linder et al. (1997) Clinical Chemistry 43:254; Marshall (1997) Nature Biotechnology 15:1249; International Patent Publication No. WO 97/40462; Schafer et al. (1998) Nature Biotechnology 16:3).

In particular, preoperative screening for the LBP alleles of this invention in a subject enables clinicians to better stratify a given patient for therapeutic intervention, either with drug therapy or with other modalities. Additionally, knowledge of LBP genotype allows patients to choose, in a more informed way in consultation with their physician, medical versus procedural therapy. Identifying the LBP genotype of patients who decide to or must undergo surgery or other invasive procedures enables health care providers to design altered therapeutic strategies aimed at preventing or minimizing the incidence of Gram negative bacterial infection in patients with the LBP allele(s) of this invention that impart increased risk. In addition, identifying the LBP genotype in patients who have already experienced Gram negative bacterial infection, or who have a relative develop Gram negative bacterial infection, might also lead to alteration or modification in the therapeutic strategy so as to be more aggressive and proactive.

As indicated above, preoperative and/or preprocedural genotype testing can refine risk stratification and improve patient outcome. Based on the genetic risk factors identified, drugs already available and used to minimize the risk of Gram negative bacterial infection (e.g., antibiotics) can be useful in reducing infection risk in acute settings, for example, in transplantation recipients. LBP genotyping can facilitate individually tailored medical therapy (personalized medicine) designed to reduce infection risk and associated morbidity and mortality. Perioperative screening can facilitate alterations in the usual course of the surgical procedure with the institution of procedures designed to additionally reduce this risk.

Thus, the present invention further provides a method of identifying an effective treatment regimen for a subject with a Gram negative bacterial infection, comprising: a) correlating the presence of one or more LBP alleles of this invention in a test subject with a Gram negative infection for whom an effective treatment regimen has been identified; and b) detecting the one or more alleles of step (a) in the subject, thereby identifying an effective treatment regimen for the subject.

Further provided is a method of correlating an LBP allele of this invention with an effective treatment regimen for Gram negative bacterial infection, comprising: a) detecting in a subject with a Gram negative bacterial infection and for whom an effective treatment regimen has been identified, the presence of one or more LBP alleles of this invention; and b) correlating the presence of the one or more alleles of step (a) with an effective treatment regimen for Gram negative bacterial infection.

Examples of treatment regimens for Gram negative bacterial infection, such as antibiotic therapy, are well known in the art.

Patients who respond well to particular treatment protocols can be analyzed for specific LBP alleles and a correlation can be established according to the methods provided herein. Alternatively, patients who respond poorly to a particular treatment regimen can also be analyzed for particular LBP alleles correlated with the poor response. Then, a subject who is a candidate for treatment for a Gram negative bacterial infection can be assessed for the presence of the appropriate LBP allele and the most appropriate treatment regimen can be provided.

In some embodiments, the methods of correlating LBP alleles with treatment regimens can be carried out using a computer database. Thus the present invention provides a computer-assisted method of identifying a proposed treatment for Gram negative bacterial infection. The method involves the steps of (a) storing a database of biological data for a plurality of patients, the biological data that is being stored including for each of said plurality of patients (i) a treatment type, (ii) at least one LBP allele associated with Gram negative bacterial infection and (iii) at least one disease progression measure for Gram negative bacterial infection from which treatment efficacy can be determined; and then (b) querying the database to determine the dependence on said LBP allele of the effectiveness of a treatment type in treating Gram negative bacterial infection, to thereby identify a proposed treatment as an effective treatment for a subject carrying an LBP allele correlated with Gram negative bacterial infection.

In one embodiment, treatment information for a patient is entered into the database (through any suitable means such as a window or text interface), LBP allele information for that patient is entered into the database, and disease progression information is entered into the database. These steps are then repeated until the desired number of patients has been entered into the database. The database can then queried to determine whether a particular treatment is effective for patients carrying a particular allele, not effective for patients carrying a particular allele, etc. Such querying can be carried out prospectively or retrospectively on the database by any suitable means, but is generally done by statistical analysis in accordance with known techniques, as described herein.

The present invention is more particularly described in the following examples that are intended as illustrative only since numerous modifications and variations therein will be apparent to those skilled in the art.

### EXAMPLES

### Methods

This study was performed using two patient populations. The first population was a retrospectively identified nested case-control population, used for identifying clinical risk factors for GN bacteremia and analysis of the association between LBP single nucleotide polymorphisms (SNP) and GN bacteremia. The second population was prospectively identified for validation of the candidate LBP SNP association with a LBP intermediate phenotype, the basal circulating LBP levels.

### Nested Case-Control Study

Patients who had their first allogeneic myeloablative HCT at the Fred Hutchinson Cancer Research Center/Seattle Cancer Care Alliance (the “Center”) between Jan. 1, 1990 and Dec. 31, 2000 and provided informed consent for the institutional review board approved genetic studies were considered for enrollment. An a priori list of GN bacterial organisms was assembled from a review of the laboratory database (Table 1). Patients were selected as a “case” if they had one or more positive blood cultures with one of these organisms prior to discharge from the Center. Control patients were selected at an approximate ratio of 2:1 to 3:1 (control:case) after meeting several criteria. Patients who did not have any positive blood cultures (due to any organisms) prior to discharge from the Center were identified as “eligible controls.” This group of “eligible controls” was further restricted by matching them to cases according to the year of transplant ±one year, then according to exposure period, defined as days to development of GN bacteremia ±10 days. Although LBP has been shown to interact with lipoteichoic acid, a cell wall component of Gram-positive bacteria such as Staphylococcus aureus and Streptococcus pneumoniae, these cases were not included, due to the desire to maintain a highly refined phenotype.¹¹ For the same reason, 30 patients who had multiorganism blood stream infections that included GN bacteria were excluded from the analysis. All cases and controls that had both patient and donor DNA available in the genetic repository were genotyped and included in the genetic analyses.¹²

Standard demographic, laboratory, and clinical data were extracted from a prospectively collected database. Disease risk categories were ranked according to the outcomes observed at the Center and have been previously described.¹³ Stem cell sources were classified as growth factor-mobilized blood cells, bone marrow, or other, which included cord blood or a combination of bone marrow and mobilized blood cells. Matching between the donor and recipient was determined according to donor-recipient HLA-A, HLA-B, and HLA-DR compatibility. Conditioning regimens were categorized as either total body irradiation based or not (containing no irradiation). To maintain a uniform at risk population, patients who received a reduced intensity conditioning regimen were excluded from this analysis. Acute and chronic graft versus host disease (GVHD) were graded based upon previously published clinical, histological, and laboratory criteria.¹⁴⁻¹⁷ Acute GVHD was categorized as present (grade 2-4) or absent (grade 0-1). Chronic GVHD was categorized according to the presence or absence of clinical extensive chronic GVHD.

Neutropenia prior to transplant was defined using the neutrophil count obtained closest to time of transplantation, prior to transplantation. Neutropenia was defined as an absolute neutrophil count (ANC) <500 cells/μl. After transplant, engraftment occurred if the ANC was ≧500 cells/μl for three consecutive days. Neutropenia after engraftment was defined as an ANC <500 cells/μl after engraftment for ≧one day. All patients with chemotherapy-induced neutropenia received systemic broad spectrum prophylactic antibiotics for bacterial prophylaxis. Blood cultures were collected for evaluation of fever (core body temperature ≧38.3° C.), and once weekly (outpatients) or twice weekly (inpatients) for patients who received systemic corticosteroids at a dose of at least 0.5 mg/kg. All patients received intermittent prophylaxis with trimethoprim/sulfamethoxazole, double-strength twice daily on Mondays and Tuesdays as first-line prophylaxis for pneumocystis pneumonia.

### Prospective Cohort and LBP Protein Measurements

Between Dec. 1, 2004 and Jan. 31, 2007, 250 patients between 18 to 65 years of age scheduled to receive an allogeneic transplant at the Center and who provided prospective consent were enrolled. Fasting whole blood was drawn, centrifuged, and the serum was aspirated and aliquoted for storage at −80° C. LBP concentrations were measured using standard ELISA techniques according to manufacturer specifications (Hycult Biotechnology, Uden, The Netherlands). Two hundred and thirty four patients who ultimately received a transplant were followed until the first episode of GN bacteremia, death or discharge from the Center through Feb. 9, 2007.

### DNA, Single Nucleotide Polymorphism Selection, and Genotyping

For the retrospective cohort, donor and recipient DNA was extracted (QIAamp DNA Blood Mini Kit, Qiagen, Valencia, Calif.) from B-lymphoblastoid cell lines immortalized by Epstein-Barr virus transformation.¹⁸ For the prospective cohort, DNA was isolated from citrated human whole blood using the Puregene DNA blood kit D-5500 (Gentra Systems, Inc. Minneapolis, Minn.).

Genetic variation data for the entire LBP gene was obtained from the Innate Immunity Program for Genomic Application, a resource that contains the full LBP gene sequence, including 5,000 base pairs up and down stream, for 23 normal European whites. From this database, 24 SNPs were identified with a minor allele frequency (MAF) ≧10% and placed in “bins” inferred according to the r² linkage disequilibrium statistic (threshold ≧0.8)¹⁹. Bin B1 included SNP 1683 (rs2232571), SNP 2111 (rs2232575), SNP 2314 (rs2232578), SNP 4507 (rs6025049), SNP 6624 (rs5741813), SNP 6662 (rs5741814), SNP 6746 (rs2232581), SNP 6878 (rs2232582), SNP 7127 (rs5741815) and SNP 11283 (rs2232590). Bin B2 included SNP 17002 (rs2232596), SNP 20012 (rs5741817), SNP 22961 (rs1739639), SNP 25253 (rs1780627), SNP 29031 (rs1780628), SNP 29556 (rs1739640) and SNP 30602 (rs1739641). Bin B3 included SNP 541 (rs1780616) and SNP 7445. Other SNPs identified were SNP 1598 (rs5741812), SNP 7400 (rs6025083), SNP 13506, SNP 28559 and SNP 33158 (rs745144).

A maximally informative tagSNP was then selected from each bin using LDSelect²⁰. This algorithm selects a subset of variants that efficiently describe all common patterns of variation in a gene, based on two primary criteria: 1) the MAF of a SNP and 2) the minimum level of association between assayed and unassayed SNPs, measured by the linkage disequilibrium statistic r². Given these parameters, LDSelect identifies bins of SNPs such that one tagSNP per bin can be genotyped. All SNPs above the MAF threshold will either be directly genotyped or will exceed the specified level of allelic association with a SNP that is genotyped. The retrospective cohort was genotyped using the Illumina Beadarray™ platform.²¹ Data quality was assessed using random duplicate samples and gender discrimination. The prospective cohort was genotyped using the ABI Taqman Assay by Design according to manufacturer specifications (Applied Biosystems, Foster City, Calif.).

### Statistical Analysis

All statistical analyses were performed using SAS (SAS Institute, Cary, N.C.), R (R Foundation), and STATA 8.0 (StataCorp, College Station, Tex.) software programs. The nested case control cohort was analyzed in two steps. In step one, clinical variables were identified that may modify the genetic effects. This analysis included all cases (N=350) and controls (N=865) and was performed by first assessing the association between each clinical variable and GN bacteremia in univariate analysis. All clinical variables that were associated with GN bacteremia at a significance level of p<0.1 were then assessed using a forward and backward stepwise selection algorithm in conditional logistic multivariate regression analysis (Table 2). Variables with at least one statistically significant category (p<0.05) in multivariate analysis were included in step two. In step two, a genetic association analysis was performed to determine if LBP tagSNPs influenced the risk for developing GN bacteremia. This analysis was restricted to cases (N=97) and controls (N=204) that had both patient and donor DNA available in the genetic repository. Patient and donor LBP tagSNPs were first assessed for deviation from Hardy-Weinberg equilibrium using a chi-squared test. Each LBP tagSNP was then independently analyzed in multivariate models, which included the clinical variables previously found to be potential effect modifiers. This analysis was performed using the Hplus software, which evaluates phenotypic association with gene-based haplotypes, while incorporating uncertainties due to unphased genotype data and adjustment for covariates.²²

For the prospective cohort, one way analysis of variance was used to assess the relationship between genotypes and log transformed LBP serum protein levels. Multivariate Cox proportional hazard regression models were used to evaluate the relationship between the presence of the putative functional SNP and time to development of GN bacteremia and death. The mortality analysis was also stratified according to presence of GN bacteremia to assess whether the effect of the putative functional SNP on mortality was more pronounced in the presence of GN bacteremia. A stepwise selection algorithm was used as above to assess pretransplant clinical variables (Table 4). The proportional hazards assumption was tested using the log-rank test.

### Results

From 3193 HCT recipients, 350 cases and 865 controls were identified. The median time to development of GN bacteremia was 53 days (range 1 to 195 days). From the univariate and multivariate analyses (Table 2), it was determined that donor gender match, disease risk, tuberculosis infection (TBI) status, cytomegalovirus (CMV) serostatus, presence of neutropenia prior to transplant and recurrence after transplant, and the development of acute and chronic GVHD, were significantly associated with GN bacteremia, and therefore, may influence the relationship between genetic variants and the risk for GN bacteremia. All of these variables were included in the subsequent LBP genetic analyses models.

Association of LBP tagSNPs and GN Bacteremia

Analysis of the LBP sequence data revealed there were 24 SNPs with a minor allele frequency ≧10%, 19 of which existed in three linkage disequilibrium bins as described above. One tagSNP from each bin was selected for genotyping: SNP 6878 (rs2232582), SNP 17002 (rs2232596), and SNP 541 (rs1780616).

From the 350 cases and 865 controls in the above epidemiologic evaluation, 97 cases and 204 controls were selected based upon availability of both patient and donor DNA samples. All patient and donor genotypes were in Hardy-Weinberg equilibrium. Univariate analysis of donor genotypes revealed no association with GN bacteremia (SNP 6878, p=0.104; SNP 17002, p=0.907; SNP 541, p=0.527), but the patient SNP 6878 genotype was significantly associated with GN bacteremia (SNP 6878, p=0.002; SNP 17002, p=0.079; SNP 541, p=0.593). Among the cases, 7 (7%) were homozygous for the SNP 6878 C allele (minor allele) and 38 (39%) were heterozygous, versus 3 (1.5%) and 55 (27%) among the controls respectively. Multivariate analysis revealed that patient SNP 6878C (p=0.001) and SNP 17002G (p=0.027) genotypes were associated with GN bacteremia (Table 3) in all participants (i.e., whites, blacks, Hispanics and Asians combined). However, in multivariate analysis restricted to whites, only the association with SNP 6878 remained significant; presence of the SNP 6878 C allele was associated with a two-fold higher risk for GN bacteremia (odds ratio=2.15, 95% confidence interval [CI], 1.31-3.52, p=0.002).

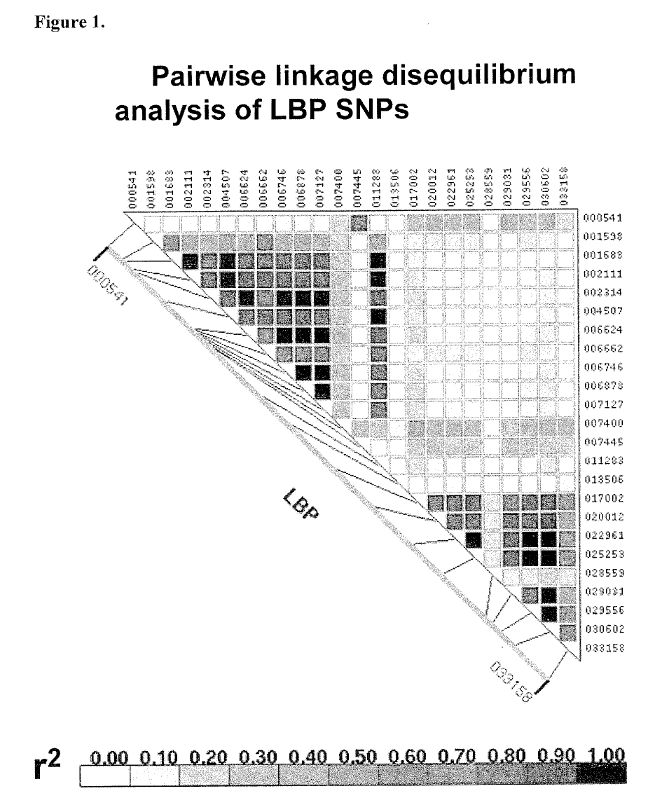

SNP 6878 tags the first LBP linkage disequilibrium bin (B1), which contains nine other SNPs. Three of the SNPs in B1, SNP 1683 (rs2232571), SNP 2111 (rs2232575), and SNP 2314 (rs2232578), map to within the 5′ 1.1-kb promoter region. SNP 6878 is in strong linkage disequilibrium with SNP 1683 (r²=1.0, FIG. 1). Based upon previous detailed mapping of the LBP promoter region, SNP 1683 confers a C/T substitution in the CAAT box at position −778.¹⁰

Association of SNP 1683 with Circulating LBP Levels and Mortality

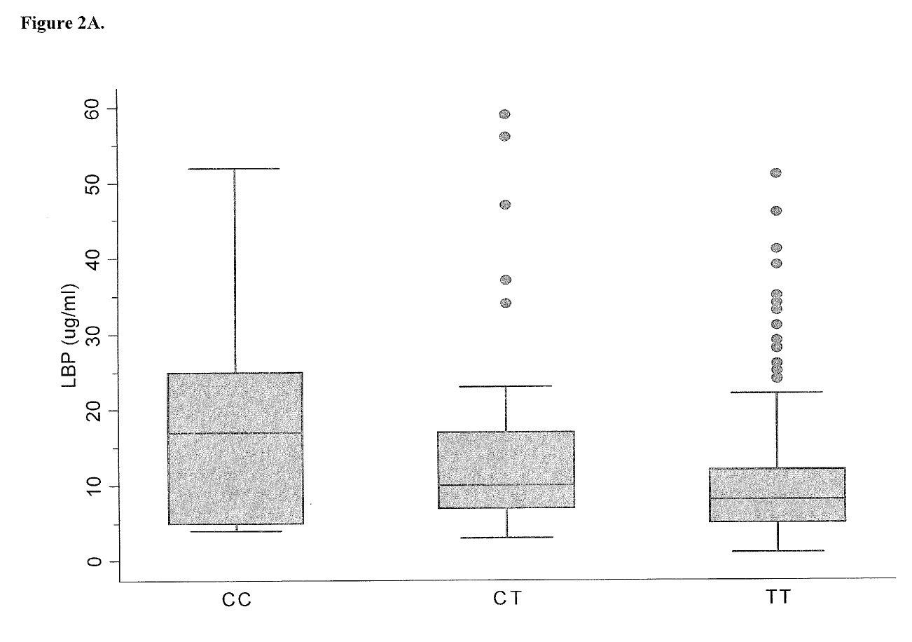

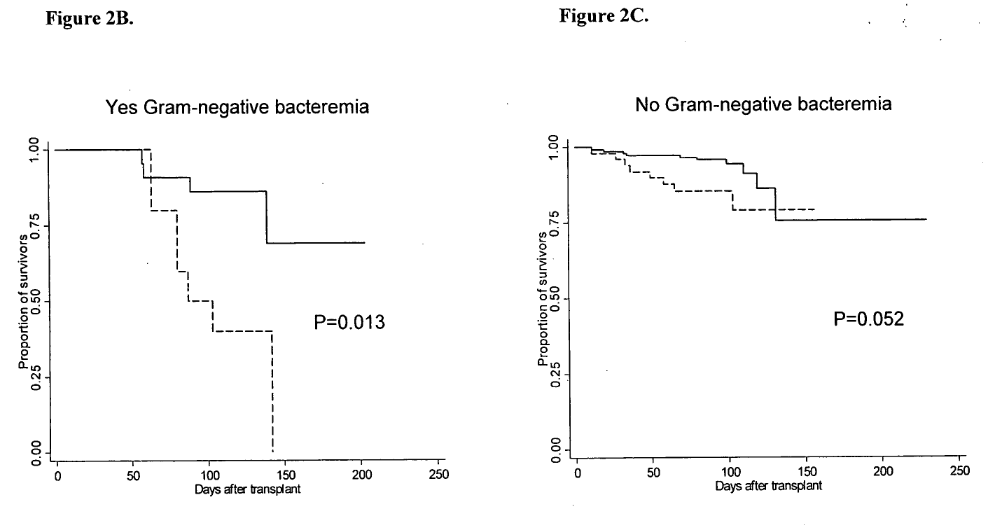

To validate the discovery data suggesting that genetic variation in the LBP gene predisposes to GN bacteremia, an assessment was made regarding whether basal LBP levels in serum collected from a prospective cohort of 250 patients being assessed for HCT correlated with the SNP 1683C genotype. SNP 1683, which was in Hardy-Weinberg equilibrium (p=0.14), was found to be significantly associated with plasma LBP levels (p=0.0036). The median plasma LBP levels according to SNP 1683 genotype were: TT (N=182) 8.07 μg/ml; TC (N=59) 10.40 μg/ml; CC (N=9) 17.39 μg/ml (FIG. 2A).

Among these patients, 234 received transplants, 32 of which developed GN bacteremia during a median follow-up time of 98 days (range 11-230 days). The SNP 1683 C allele was significantly associated with an overall 3-fold increase in risk of death prior to discharge from the Center (hazard ratio [HR]=3.30, 95% CI, 1.59-6.84, p=0.001; Table 5). When this analysis was stratified according to GN bacteremia status, patients with the SNP 1683 C allele who developed GN bacteremia had a significant 5-fold increase in mortality risk (HR=4.83, 95% CI, 1.38-16.75, p=0.013; FIGS. 2B-C); among patients with GN bacteremia, 64% (N=7) of those who died had the SNP 1683 C allele, versus 14% (N=3) among those who survived. Even among patients who did not develop GN bacteremia, patients with the SNP 1683 C allele had a borderline significant 2-fold increase in mortality risk (HR 2.51, 95% CI 0.99-6.37, p=0.052). Only older patient age was associated with death after transplant in univariate analysis (p=0.019; Table 4). However, patient age was not a significant factor in the multivariate analysis. The SNP 1683 C allele was not significantly associated with an increase in risk for GN bacteremia, but this was expected because the size of the prospective cohort was not designed to detect an association with GN bacteremia.

These results demonstrate that genetic variation in the promoter region of the LBP gene is associated with the blood level of LBP and with the risk of developing GN bacteremia and GN bacteremia-related death after HCT. Transcriptional activity of the LBP gene is partly governed by the patient genotype. SNP 1683 confers a C/T substitution at position −778, which is located in one of the LBP CAAT boxes, which are transcriptional elements that regulate the efficiency of the promoter. In promoter truncation experiments that excluded this region, LBP promoter inducibility increased three-fold in comparison to when the entire promoter was intact.¹⁰ Association of the patients' SNP 1683 genotype with a two-fold higher LBP level suggests that presence of the minor SNP 1683 C allele may enhance the efficiency of the promoter. Unlike previous candidate LBP SNP approaches,^{23,24} this study benefited from knowledge of the genetic variation across the entire LBP gene. Nearly 80% of all the common LBP SNPs, defined as SNPs with a minor allele frequency ≧10%, were analyzed by genotyping for only three tagSNPs. In the context of a biologically relevant phenotype and a racially uniform population, this maximized the likelihood of finding a meaningful genetic association.

The association of SNP 1683C with a five-fold increase in risk of death after transplant among patients with GN bacteremia, and a borderline effect among patients without GN bacteremia, suggests several possible mechanisms by which LBP variants might influence mortality risk. This finding is consistent with the biology of LBP and the major role it plays in modulating the host immune response to GN bacteria and LPS.²⁵ If high levels of LBP down regulate the innate immune response to GN bacteria, which is essentially the only immune response available during the early post transplant period, the outcome may be disastrous in the presence of GN bacteremia. The borderline association observed among patients without GN bacteremia may be related to clinically undetectable GN bacteremia. Due to intestinal mucosal damage related to the conditioning regimen, GN bacteria commonly translocate across the intestinal mucosa during the early post-transplant period.²⁶ In the setting of a genetically predisposed patient whose LBP levels are high, this relatively low level of bacteremia that is undetectable by standard clinical techniques may be allowed to advance unchecked by the innate immune system, ultimately leading to increased mortality. The magnitude of the genetic attributable risk is also noteworthy. The risk for mortality associated with SNP 1683 is higher than nearly all clinical predictors of mortality recently identified in a multi-stage cohort study of over 2400 patients.²⁷ These results suggest the use of SNP 1683 as a predictor of mortality risk.

The foregoing is illustrative of the present invention, and is not to be construed as limiting thereof. The invention is defined by the following claims, with equivalents of the claims to be included therein. All publications, patent applications, patents, patent publications, sequences (nucleotide sequences, single polymorphism nucleotides, amino acid sequences, etc.) identified in the GenBank® database or other sequence databases according to the accession numbers provided herein, and any other references cited herein are incorporated by reference in their entireties for the teachings relevant to the sentence and/or paragraph in which the reference is presented.

### REFERENCE LIST

1. Gereda J E, Klinnert M D, Price M R, Leung D Y, Liu A H. Metropolitan home living conditions associated with indoor endotoxin levels. J Allergy Clin Immunol. 2001; 107:790-796.
2. Heinrich J, Gehring U, Douwes J, et al. Pets and vermin are associated with high endotoxin levels in house dust. Clin Exp Allergy. 2001; 31:1839-1845.
3. Zweigner J, Schumann R R, Weber J R. The role of lipopolysaccharide-binding protein in modulating the innate immune response. Microbes & Infection. 2006; 8:946-952.
4. Wright S D, Ramos R A, Tobias P S, Ulevitch R J, Mathison J C. CD14, a receptor for complexes of lipopolysaccharide (LPS) and LPS binding protein. Science. 1990; 249:1431-1433.
5. Schumann R R, Leong S R, Flaggs G W, et al. Structure and function of lipopolysaccharide binding protein. Science. 1990; 249:1429-1431.
6. Martin T R, Mathison J C, Tobias P S, et al. Lipopolysaccharide binding protein enhances the responsiveness of alveolar macrophages to bacterial lipopolysaccharide. J Clin Invest. 1992; 90:2209-2219.
7. Le Roy D, Di Padova F, Adachi Y, Glauser M P, Calandra T, Heumann D. Critical role of lipopolysaccharide-binding protein and CD14 in immune responses against Gram-negative bacteria. J Immunol. 2001; 167:2759-2765.
8. Zweigner J, Gramm H J, Singer O C, Wegscheider K, Schumann R R. High concentrations of lipopolysaccharide-binding protein in serum of patients with severe sepsis or septic shock inhibit the lipopolysaccharide response in human monocytes. Blood. 2001; 98:3800-3808.
9. Lamping N, Dettmer R, Schroder N W, et al. LPS-binding protein protects mice from septic shock caused by LPS or Gram-negative bacteria. J Clin Invest. 1998; 101:2065-2071.
10. Schumann R R, Kirschning C J, Unbehaun A, et al. The lipopolysaccharide-binding protein is a secretory class 1 acute-phase protein whose gene is transcriptionally activated by APRF/STAT/3 and other cytokine-inducible nuclear proteins. Mol Cell Biol. 1996; 16:3490-3503.
11. Mueller M, Stamme C, Draing C, Hartung T, Seydel U, Schromm A B. Cell activation of human macrophages by lipoteichoic acid is strongly attenuated by lipopolysaccharide-binding protein. J Biol Chem. 2006; 281:31448-31456.
12. Schroder N W, Morath S, Alexander C, et al. Lipoteichoic acid (LTA) of Streptococcus pneumoniae and Staphylococcus aureus activates immune cells via Toll-like receptor (TLR)-2, lipopolysaccharide-binding protein (LBP), and CD14, whereas TLR-4 and MD-2 are not involved. J Biol Chem. 2003; 278:15587-15594.
13. Chien J W, Zhao L P, Hansen J A, Fan W H, Parimon T, Clark J G. Genetic variation in bactericidal/permeability-increasing protein influences the risk of developing rapid airflow decline after hematopoietic cell transplantation. Blood. 2006; 107:2200-2207.
14. Shulman H M, Sullivan K M, Weiden P L, et al. Chronic graft-versus-host syndrome in man. A long-term clinicopathologic study of 20 Seattle patients. Am J Med. 1980; 69:204-217.
15. Przepiorka D, Smith T L, Folloder J, et al. Risk factors for acute graft-versus-host disease after allogeneic blood stem cell transplantation. Blood. 1999; 94:1465-1470.
16. Sullivan K M. Graft-versus-host disease. In: Thomas E D, Blume K G, Forman S J, eds. Hematopoietic Cell Transplantation. Malden, Mass.: Blackwell Sciences, Inc.; 1999:515-536.
17. Sullivan K M, Shulman H M, Storb R, et al. Chronic graft-versus-host disease in 52 patients: adverse natural course and successful treatment with combination immunosuppression. Blood. 1981; 57:267-276.
18. Neitzel H. A routine method for the establishment of permanent growing lymphoblastoid cell lines. Hum Genet. 1986; 73:320-326.
19. Devlin B, Risch N. A comparison of linkage disequilibrium measures for fine-host mapping. Genomics. 1995; 29:311-322.
20. Carlson C S, Eberle M A, Rieder M J, Yi Q, Kruglyak L, Nickerson D A. Selecting a maximally informative set of single-nucleotide polymorphisms for association analyses using linkage disequilibrium. Am J Hum Genet. 2004; 74:106-120.
21. Fan J B, Oliphant A, Shen R, et al. Highly parallel SNP genotyping. Cold Spring Harbor Symp Quant Biol. 2003; 68:69-78.
22. Zhao L P, Li S S, Khalid N. A method for the assessment of disease associations with single-nucleotide polymorphism haplotypes and environmental variables in case-control studies. Am J Hum Genet. 2003; 72:1231-1250.
23. Hubacek J A, Stuber F, Frohlich D, et al. Gene variants of the bactericidal/permeability increasing protein and lipopolysaccharide binding protein in sepsis patients: gender-specific genetic predisposition to sepsis. Crit Care Med. 2001; 29:557-561.
24. Hubacek J A, Pitha J, Skodova Z, et al. Polymorphisms in the lipopolysaccharide-binding protein and bactericidal/permeability-increasing protein in patients with myocardial infarction. Clinical Chemistry & Laboratory Medicine. 2002; 40:1097-1100.
25. Ferrara J L. Pathogenesis of acute graft-versus-host disease: cytokines and cellular effectors (Review). J Hematother Stem Cell Res. 2000; 9:299-306.
26. Hill G R, Crawford J M, Cooke K R, Brinson Y S, Pan L, Ferrara J L. Total body irradiation and acute graft-versus-host disease: the role of gastrointestinal damage and inflammatory cytokines. Blood. 1997; 90:3204-3213.
27. Parimon T, Au D H, Martin P J, Chien J W. A risk score for mortality after allogeneic hematopoietic cell transplantation. Ann Intern Med. 2006; 144:407-414.

TABLE 1
Causes of Gram-negative bacteremia after allogeneic
hematopoietic cell transplant between 1990 and 2006.

Acinetobacter alcaligenes
Acinetobacter baumannii
Acinetobacter calcoaceticus var. anitratus
Acinetobacter calcoaceticus var. lwoffi
Acinetobacte rursingii
Acinetobacter, NOS
Aeromonas caviae
Aeromonas hydrophila
Agrobacterium tumefaciens
Alcaligenes, NOS
Bacteroides distasonis
Burkholderia cepacia
Citrobacter freundii
Enterobacter aerogenes
Enterobacter agglomerans
Enterobacter asburiae
Enterobacter cloacae
Enterobacter, NOS
Escherichia coli
Klebsiella oxytoca
Klebsiella ozenae
Kebsiella pneumoniae
Kebsiella, NOS
Leptotrichia, NOS
Morganella morganii
Moraxella catarrhalis
Moraxella osloensis
Moraxella, NOS
Neisseria sicca
Neisseria, NOS
Pantoea agglomerans
Proteus mirabilis
Pseudomonas acidvorans
Pseudomonas aeruginosa
Pseudomonas cepacia
Pseudomonas diminuta
Pseudomonas fluorescens
Pseudomonas maltophilia
Pseudomonas orzihabitans
Pseudomonas paucimobilis
Pseudomonas putida
Pseudomonas stutzeri
Pseudomonas veriscularis
Pseudomonas, NOS
Ralstonia pickettii
Serratia liquefaciens
Serratia marcescens
Serratia, NOS
Stenotrophomonas maltophilia

TABLE 2
Comparison of the clinical characteristics of patients who developed Gram-negative
bacteremia with patients who did not develop bacteremia.

| | Univariate analysis | | | Multivariate analysis | |
|---|---|---|---|---|---|
| | Controls | Cases | | Odds ratio | |
| Clinical Variables | (N = 865) | (N = 350) | P-value | (95% CI) | P-value |
| Age (years) | 34.0 ± 15.2 | 34.1 ± 15.9 | 0.95 | — | — |

Sex match (P:D)

| | | | | | | | |
|---|---|---|---|---|---|---|---|
| Male:male | 305 | (35) | 109 | (31) | 0.024 | Referent | — |
| Male:female | 196 | (23) | 72 | (21) | | 0.95 (0.65-1.40) | 0.791 |
| Female:male | 194 | (22) | 108 | (31) | | 1.51 (1.06-2.16) | 0.023 |
| Female:female | 169 | (20) | 61 | (17) | | 1.01 (0.67-1.50) | 0.977 |

Race match (P:D)

| | | | | | | | |
|---|---|---|---|---|---|---|---|
| White:white | 765 | (88) | 294 | (84) | 0.105 | — | — |
| White:nonwhite | 7 | (1) | 4 | (1) | | | |
| Nonwhite:white | 8 | (1) | 8 | (2) | | | |
| Nonwhite:nonwhite | 85 | (10) | 44 | (13) | | | |

Disease risk

| | | | | | | | |
|---|---|---|---|---|---|---|---|
| Low | 298 | (35) | 93 | (26) | 0.015 | Referent | — |
| Moderate | 237 | (27) | 97 | (28) | | 0.99 (0.68-1.45) | 0.974 |
| High | 330 | (38) | 160 | (46) | | 1.43 (1.00-2.04) | 0.049 |

Donor type

| | | | | | | | |
|---|---|---|---|---|---|---|---|
| Matched related | 452 | (52) | 156 | (45) | 0.043 | — | — |
| Mismatched related | 103 | (12) | 53 | (15) | | | |
| Unrelated | 310 | (36) | 141 | (40) | | | |

Total body irradiation

| | | | | | | | |
|---|---|---|---|---|---|---|---|
| No | 335 | (39) | 84 | (24) | <0.001 | Referent | — |
| Yes | 530 | (61) | 266 | (76) | | 1.50 (1.08-2.07) | 0.015 |

Stem cell source

| | | | | | | | |
|---|---|---|---|---|---|---|---|
| Bone marrow | 762 | (88) | 326 | (93) | 0.024 | — | — |
| PBSC | 96 | (11) | 21 | (6) | | | |
| Other | 7 | (1) | 3 | (1) | | | |

CMV serostatus (P:D)

| | | | | | | | |
|---|---|---|---|---|---|---|---|
| Negative:negative | 397 | (46) | 100 | (29) | <0.001 | Referent | — |
| Negative:positive | 152 | (18) | 54 | (15) | | 1.39 (0.93-2.09) | 0.113 |
| Positive:negative | 125 | (14) | 91 | (26) | | 2.59 (1.77-3.81) | <0.001 |
| Positive:positive | 188 | (22) | 104 | (30) | | 2.57 (1.78-3.69) | <0.001 |

Pretransplant neutropenia

| | | | | | | | |
|---|---|---|---|---|---|---|---|
| No | 441 | (51) | 132 | (38) | <0.001 | Referent | — |
| Yes | 424 | (49) | 218 | (62) | | 1.37 (1.01-1.87) | 0.045 |

| | | | | | |
|---|---|---|---|---|---|
| Days to engraftment | 20.5 ± 5.2 | 20.0 ± 5.8 | 0.127 | — | — |

Neutropenia after initial engraftment

| | | | | | | | |
|---|---|---|---|---|---|---|---|
| No | 783 | (91) | 246 | (70) | <0.001 | Referent | — |
| Yes | 82 | (9) | 104 | (30) | | 2.77 (1.95-3.95) | <0.001 |

Acute GVHD

| | | | | | | | |
|---|---|---|---|---|---|---|---|
| No | 334 | (39) | 55 | (16) | <0.001 | Referent | — |
| Yes | 518 | (61) | 292 | (84) | | 3.03 (2.14-4.27) | <0.001 |

Chronic GVHD

| | | | | | | | |
|---|---|---|---|---|---|---|---|
| No | 574 | (67) | 183 | (55) | <0.001 | Referent | — |
| Yes | 284 | (33) | 155 | (45) | | 1.66 (1.24-2.22) | 0.001 |

CI = confidence interval; P = patient; D = donor; PBSC = peripheral blood stem cell; GVHD = graft versus host disease

TABLE 3
Association of patient LBP tagSNP genotypes with GN bacteremia

| | All participants (97 cases. 204 controls) | | | | Whites only (85 cases, 189 controls) | | | |
|---|---|---|---|---|---|---|---|---|
| | Allele | | | | Allele | | | |
| | frequencies | | Odds ratio | | frequencies | | Odds ratio | |
| tagSNP | Case | Control | (95% CI) | P-value | Case | Control | (95% CI) | P-value |
| 6878 (C/T) | 0.26 | 0.15 | 2.22 (1.39-3.56) | 0.001 | 0.27 | 0.16 | 2.15 (1.31-3.52) | 0.002 |
| 17002 (A/G) | 0.40 | 0.50 | 0.65 (0.44-0.95) | 0.027 | 0.41 | 0.49 | 0.7 (0.47-1.06) | 0.089 |
| 541 (C/T) | 0.34 | 0.32 | 0.93 (0.63-1.38) | 0.729 | 0.32 | 0.34 | 0.9 (0.6-1.35) | 0.602 |

Each tagSNP was analyzed in independent multivariate models that included gender match, disease risk, TBI dose, CMV serostatus, presence of neutropenia pretransplant, recurrent neutropenia after engraftment, and presence of acute or chronic GVHD as covariates.

TABLE 4
Pretransplant clinical characteristics of the prospective cohort
compared according to Gram-negative bacteremia and mortality

| | Gram-negative bacteremia | | | Death | | |
|---|---|---|---|---|---|---|
| | No | Yes | | No | Yes | |
| Clinical Variables | (N = 218) | (N = 32) | P-value | (N = 205) | (N = 29) | P-value |
| Age (years) | 49.72 ± 13.36 | 50.05 ± 13.75 | 0.897 | 48.98 ± 13.13 | 55.29 ± 14.11 | 0.017 |

Sex match (P:D)

| | | | | | | | | | | |
|---|---|---|---|---|---|---|---|---|---|---|
| Male:male | 76 | (35) | 10 | (31) | 0.837 | 63 | (31) | 7 | 24) | 0.519 |
| Male:female | 64 | (29) | 8 | (25) | | 59 | (29) | 13 | (45) | |
| Female:male | 42 | (19) | 7 | (22) | | 43 | (21) | 6 | (21) | |
| Female:female | 36 | (17) | 7 | (22) | | 40 | (19) | 3 | (10) | |

Disease risk

| | | | | | | | | | | |
|---|---|---|---|---|---|---|---|---|---|---|
| Low | 16 | (8) | 2 | (6) | 0.942 | 16 | (8) | 2 | (7) | 0.157 |
| Moderate | 101 | (50) | 16 | (50) | | 107 | (52) | 10 | (34) | |
| High | 85 | (42) | 14 | (44) | | 82 | (40) | 17 | (59) | |

Donor type

| | | | | | | | | | | |
|---|---|---|---|---|---|---|---|---|---|---|
| Related | 80 | (40) | 15 | (47) | 0.693 | 87 | (42) | 8 | (28) | 0.281 |
| Unrelated | 121 | (60) | 17 | (53) | | 1 | (<1) | 0 | (0) | |
| ISO | 1 | (<1) | 0 | (0) | | 117 | (57) | 21 | (72) | |

Conditioning regimen

| | | | | | | | | | | |
|---|---|---|---|---|---|---|---|---|---|---|
| Nonmyeloablative | 90 | (45) | 19 | (59) | 0.278 | 93 | (45) | 16 | (55) | 0.449 |

Myeloablative

| | | | | | | | | |
|---|---|---|---|---|---|---|---|---|
| No TBI | 75 | (37) | 8 | (25) | 39 | (19) | 3 | (10) |
| Yes TBI | 37 | (18) | 5 | (16) | 73 | (36) | 10 | (35) |

Stem cell source

| | | | | | | | | | | |
|---|---|---|---|---|---|---|---|---|---|---|
| Bone marrow | 24 | (12) | 7 | (22) | 0.189 | 27 | (13) | 4 | (14) | 0.600 |
| PBSC | 171 | (85) | 25 | (78) | | 171 | (83) | 25 | (86) | |
| Cord | 7 | (3) | | 0 | | 7 | (4) | 7 | (3) | |

CMV serostatus (P:D)

| | | | | | | | | | | |
|---|---|---|---|---|---|---|---|---|---|---|
| Negative:negative | 86 | (39) | 11 | (34) | 0.353 | 71 | (35) | 10 | (34) | 0.663 |
| Negative:positive | 19 | (9) | 6 | (19) | | 20 | (10) | 5 | (17) | |
| Positive:negative | 66 | (30) | 8 | (25) | | 66 | (32) | 8 | (28) | |
| Positive:positive | 47 | (22) | 7 | (22) | | 48 | (23) | 6 | (21) | |

Pretransplant neutropenia

| | | | | | | | | | | |
|---|---|---|---|---|---|---|---|---|---|---|
| No | 186 | (85) | 25 | (78) | 0.295 | 174 | (85) | 21 | (72) | 0.092 |
| Yes | 32 | (15) | 7 | (22) | | 313 | (15) | 8 | (28) | |

P = patient; D = donor; TBI = total body irradiation ≧1200 Gy; PBSC = peripheral blood stem cell

TABLE 5
Relation ship between SNP 1683 and mortality
after transplant in the prospective cohort

| | |
|---|---|
| SNP 1683 | Death N (%) |

| genotype | No | Yes | Total |
|---|---|---|---|
| TT | 158 (92) | 14 (8) | 172 |
| TC | 41 (76) | 13 (24) | 54 |
| CC | 6 (75) | 2 (25) | 8 |

p = 0.004

That which is claimed is:

1. A method of identifying a subject as having an increased risk of developing a Gram negative bacterial infection, comprising genotyping the subject for the presence of a C allele of the single nucleotide polymorphism rs2232582 of the lipopolysaccharide binding protein gene, wherein the presence of said C allele identifies the subject as having an increased risk of developing a Gram negative bacterial infection.

2. A method of identifying a subject as having an increased risk of developing a Gram negative bacterial infection, comprising genotyping the subject for the presence of a C allele of the single nucleotide polymorphism rs2232571 of the lipopolysaccharide binding protein gene, wherein the presence of said C allele identifies the subject as having an increased risk of developing a Gram negative bacterial infection.

3. The method of claim 1, wherein the subject is a high risk subject.

4. The method of claim 2, wherein the subject is a high risk subject.

5. A method of identifying a subject as having an increased risk of mortality, comprising genotyping the subject for the presence of a C allele of the single nucleotide polymorphism rs2232582 of the lipopolysaccharide binding protein gene, wherein the presence of said C allele identifies the subject as having an increased risk of mortality.

6. A method of identifying a subject as having an increased risk of mortality, comprising genotyping the subject for the presence of a C allele of the single nucleotide polymorphism rs2232571 of the lipopolysaccharide binding protein gene, wherein the presence of said C allele identifies the subject as having an increased risk of mortality.

7. The method of claim 5, wherein the subject is a high risk subject.

8. The method of claim 6, wherein the subject is a high risk subject.

9. A method of identifying a subject as having an increased risk of developing a Gram negative bacterial infection, comprising genotyping the subject for the presence of an allele of a single nucleotide polymorphism of the lipopolysaccharide binding protein gene of the subject, wherein the allele is selected from the group consisting of:
a) a C allele of the single nucleotide polymorphism rs2232571;
b) a C allele of the single nucleotide polymorphism rs2232582;
c) a C allele of the single nucleotide polymorphism rs2232575;
d) a G allele of the single nucleotide polymorphism rs2232578;
e) an A allele of the single nucleotide polymorphism rs6025049;
f) a G allele of the single nucleotide polymorphism rs5741813;
g) a T allele of the single nucleotide polymorphism rs5741814;
h) a G allele of the single nucleotide polymorphism rs2232581;
i) a C allele of the single nucleotide polymorphism rs5741815;
j) a G allele of the single nucleotide polymorphism rs2232590; and
h) any combination thereof,
wherein the presence of said allele or combination of alleles identifies the subject as having an increased risk of developing a Gram negative bacterial infection.

10. The method of claim 9, wherein the subject is a high risk subject

11. A method of identifying a subject as having an increased risk of mortality, comprising genotyping the subject for the presence of an allele of a single nucleotide polymorphism of the lipopolysaccharide binding protein gene of the subject, wherein the allele is selected from the group consisting of:
a) a C allele of the single nucleotide polymorphism rs2232571;
b) a C allele of the single nucleotide polymorphism rs2232582;
c) a C allele of the single nucleotide polymorphism rs2232575;
d) a G allele of the single nucleotide polymorphism rs2232578;
e) an A allele of the single nucleotide polymorphism rs6025049;
f) a G allele of the single nucleotide polymorphism rs5741813;
g) a T allele of the single nucleotide polymorphism rs5741814;
h) a G allele of the single nucleotide polymorphism rs2232581;
i) a C allele of the single nucleotide polymorphism rs5741815;
j) a G allele of the single nucleotide polymorphism rs2232590; and
h) any combination thereof,
wherein the presence of said allele or combination of alleles identifies the subject as having an increased risk of mortality.

12. The method of claim 11, wherein the subject is a high risk subject.

13. The method of claim 1, wherein the Gram negative bacterial infection is in the blood of the subject.

14. A method of screening for increased risk of a Gram negative bacterial infection or increased mortality in a subject, wherein the presence of an allele in the lipopolysaccharide binding protein gene of the subject selected from the group consisting of:
a) a C allele of the single nucleotide polymorphism rs2232571;
b) a C allele of the single nucleotide polymorphism rs2232582;
c) a C allele of the single nucleotide polymorphism rs2232575;
d) a G allele of the single nucleotide polymorphism rs2232578;
e) an A allele of the single nucleotide polymorphism rs6025049;
f) a G allele of the single nucleotide polymorphism rs5741813;
g) a T allele of the single nucleotide polymorphism rs5741814;
h) a G allele of the single nucleotide polymorphism rs2232581;
i) a C allele of the single nucleotide polymorphism rs5741815;
j) a G allele of the single nucleotide polymorphism rs2232590; and
h) any combination thereof, indicates said subject is at increased risk of a Gram negative bacterial infection or increased mortality, comprising detecting the presence or absence of said allele(s) in a biological sample of said subject.

15. The method of claim 14, wherein the subject is a high risk subject.

16. The use of a means of detecting an allele of a lipopolysaccharide binding protein, wherein said allele is selected from the group consisting of:
a) a C allele of the single nucleotide polymorphism rs2232571;
b) a C allele of the single nucleotide polymorphism rs2232582;
c) a C allele of the single nucleotide polymorphism rs2232575;
d) a G allele of the single nucleotide polymorphism rs2232578;
e) an A allele of the single nucleotide polymorphism rs6025049;
f) a G allele of the single nucleotide polymorphism rs5741813;
g) a T allele of the single nucleotide polymorphism rs5741814;
h) a G allele of the single nucleotide polymorphism rs2232581;
i) a C allele of the single nucleotide polymorphism rs5741815;
j) a G allele of the single nucleotide polymorphism rs2232590; and
h) any combination thereof,
in a biological sample of a subject, in determining if said subject is at increased risk of a Gram negative bacterial infection or mortality.

17. The use of claim 16, wherein the subject is a high risk subject.
